(19) United States
(12) Patent Application Publication
(10) Pub. No.: US 2016/0103110 A1
(43) Pub. Date: Apr. 14, 2016

(54) ENGINE NOX MODEL

(71) Applicants: Adam C. Lack, Boulder, CO (US); Navtej Singh, Arlington Heights, IL (US); Prasanna Nagabushan-Venkatesh, Lombard, IL (US)

(72) Inventors: Adam C. Lack, Boulder, CO (US); Navtej Singh, Arlington Heights, IL (US); Prasanna Nagabushan-Venkatesh, Lombard, IL (US)

(73) Assignee: International Engine Intellectual Property Company, LLC, Lisle, IL (US)

(21) Appl. No.: 14/893,373

(22) PCT Filed: May 24, 2013
(86) PCT No.: PCT/US13/42753

§ 371 Date: Nov. 23, 2015

(51) Int. Cl. G01N 33/00; G01M 15/10

(52) U.S. Cl. CPC G01N 33/0037; G01M 15/106

(57) ABSTRACT

A method is provided for estimating the NOₓ content of exhaust gas produced by an internal combustion engine. The method includes determining a first NOₓ estimate corresponding to the NOₓ level output by the engine during a first engine operating condition. The method further includes determining a second NOₓ estimate corresponding to the NOₓ level output by the engine during a second engine operating condition. The method further includes determining a compensation factor based on intake manifold pressure and applying the compensation factor to the first and second NOₓ estimates to arrive at a final NOₓ estimate.

### BACKGROUND

Selective catalytic reduction (SCR) is commonly used to remove NOₓ (i.e., oxides of nitrogen) from the exhaust gas produced by internal engines, such as diesel or other lean burn (gasoline) engines. In such systems, NOₓ is continuously removed from the exhaust gas by injection of a reductant into the exhaust gas prior to entering an SCR catalyst capable of achieving a high conversion of NOₓ.

Ammonia is often used as the reductant in SCR systems. The ammonia is introduced into the exhaust gas by controlled injection either of gaseous ammonia, aqueous ammonia or indirectly as urea dissolved in water. The SCR catalyst, which is positioned in the exhaust gas stream, causes a reaction between NOₓ present in the exhaust gas and a NOₓ reducing agent (e.g., ammonia) to convert the NOₓ into nitrogen and water.

Proper operation of the SCR system involves precise control of the amount (i.e., dosing level) of ammonia (or other reductant) that is injected into the exhaust gas stream. Injection of too much reductant causes a slip of ammonia in the exhaust gas, whereas injection of a too little reductant causes a less than optimal conversion of NOₓ. Thus, SCR systems often utilize NOₓ sensors in order to determine proper reactant dosing levels. For example, a NOₓ sensor can be positioned in the exhaust stream between the engine and the SCR catalyst for detecting the level of NOₓ that is being emitted from the engine. This is commonly referred to as an engine out NOₓ sensor or an upstream NOₓ sensor. An electronic control unit (ECU) can use the output from the engine out NOₓ sensor (and/or other sensed parameters) to determine the amount of reductant that should to be injected into the exhaust stream.

Commercially, available NOₓ sensors are expensive and have other operation drawbacks. For example, the accuracy of NOₓ sensors can be affected by environmental and/or operating conditions such as dew point, system voltage, oxygen concentration and the like. In this regard, some NOₓ only work properly when the exhaust gas is above a threshold temperature which can be on the order of 125-130° C. As a result, such sensors may not suitable for determining dosing levels during certain engine operating conditions, such as low idle or engine warm-up. Hence, it is desirable to provide an alternative method for determining the NOₓ level in an engine's exhaust.

### SUMMARY

Aspects and embodiments of the present technology described herein relate to one or more systems and methods for controlling the operation of an engine. According to at least one aspect of the present technology, at least one method is provided for estimating the NOₓ content of exhaust gas produced by an internal combustion engine. The estimated NOₓ level can be used by a control unit for controlling operation of an SCR system, for example. The method includes determining a first NOₓ estimate corresponding to the NOₓ level output by the engine during a first engine operating condition. The method further includes determining a second NOₓ estimate corresponding to the NOₓ level output by the engine during a second engine operating condition. The method further includes determining a compensation factor based on intake manifold pressure and applying the compensation factor to the first and second NOₓ estimates to arrive at a final NOₓ estimate.

According to certain aspects of the present technology, the first and second NOₓ estimates are each determined as a function of at least engine speed and torque. In at least one embodiment, the first engine operating condition corresponds to substantially steady state engine operation where the engine is operating a substantially constant speed, while the second engine operating condition corresponds to a transitory engine operation where engine power is increasing.

According to certain aspects of the present technology, the step of determining a compensation factor further includes the steps of determining an estimated intake manifold pressure as a function of engine speed and torque, sensing the actual intake manifold pressure, and determining the compensation factor as a function of the actual and estimated intake manifold pressures. In at least one embodiment, the compensation factor is determined as a function of a difference between the actual and estimated intake manifold pressures. In some embodiments, the compensation factor ranges from 0 to 1 and increases as the difference between the actual and estimated manifold pressure increases.

According to further aspects of the present technology, the compensation factor is also a function one or more of exhaust manifold pressure, mass air flow, turbocharger boost, exhaust flow. In some embodiments, the estimated level of NOₓ output by the engine (NOₓ_{_}EST_OUT) is determined in accordance with the following formula:

NOₓ_{_}OUT_EST=(CF•NOₓ_{_}T)+((1−CF)•NOₓ_{_}SS)

where CF is the compensation factor, NOₓ_{_}SS is the first NOₓ estimate and NOₓ_{_}T is the second NOₓ estimate.

According to at least one aspect of the present technology, at least one method is provided for estimating the NOₓ content of exhaust gas produced by an internal combustion engine. The method includes determining a steady state NOₓ estimate as a function of at least engine speed and torque. The steady state NOₓ corresponds to the NOₓ level output by the engine during a substantially steady state operation where engine speed and power are substantially constant. The method further includes determining a transitory NOₓ estimate as a function of at least engine speed and torque. The transitory NOₓ estimate corresponds to the NOₓ level output by the engine during a transitory operation where engine power is increasing. The method also includes determining a compensation factor based on intake manifold pressure and applying the compensation factor to the steady state and transitory NOₓ estimates to arrive at a final NOₓ estimate. In some embodiments, the compensation factor weights the final NOₓ estimate towards the transitory NOₓ estimate with decreasing intake manifold pressure.

According to another aspect of at least one embodiment of the present technology, a method for estimating the NOₓ content of exhaust gas produced by an internal combustion engine includes determining a steady state NOₓ estimate (NOₓ_{_}SS) as a function of at least engine speed and torque. The steady state NO corresponds to the NOₓ level output by the engine during a substantially steady state operation where engine speed and power are substantially constant. The method also includes determining a transitory NOₓ estimate (NOₓ_{_}T) as a function of at least engine speed and torque. The transitory NOₓ estimate corresponding to the NOₓ level output by the engine during a transitory operation where engine power is increasing. The method further includes determining an estimated intake manifold pressure as a function of at least engine speed and torque. The method also includes sensing the actual intake manifold pressure and determining the compensation factor (CF) as a function of a difference between the actual and estimated intake manifold pressures. According to at least one aspect of the present technology, the compensation factor has a value ranging from 0 to 1 and increases as the difference between the actual and estimated intake manifold pressures increases. The method also includes determining final NOx estimate (NOₓ_{_}OUT_EST) in accordance with the following formula:

NOₓ_{_}OUT_EST=(CF•NOₓ_{_}T)+((1−CF)•NOₓ_{_}SS).

### BRIEF DESCRIPTION OF THE DRAWINGS

FIG. 1 is a schematic illustration of an internal combustion engine with an exhaust gas SCR system.

FIG. 2 is a flow diagram of an exemplary method for determining the NO level in an engine's exhaust.

FIG. 3 is a schematic of exemplary control logic for determining the NO level in an engine's exhaust.

### DETAILED DESCRIPTION

Various examples of embodiments of the present technology will be described more fully hereinafter with reference to the accompanying drawings, in which such examples of embodiments are shown. Like reference numbers refer to like elements throughout. Other embodiments of the presently described technology may, however, be in many different forms and are not limited solely to the embodiments set forth herein. Rather, these embodiments are examples representative of the present technology. Rights based on this disclosure have the full scope indicated by the claims.

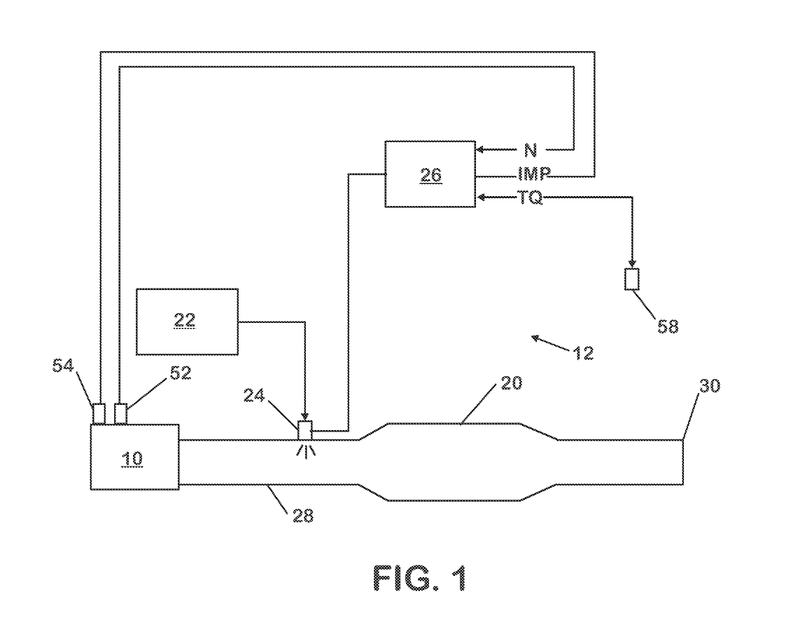

FIG. 1 shows an exemplary schematic depiction of an internal combustion engine 10 and an SCR system 12 for reducing NOₓ from the engine's exhaust. The engine can be used, for example, to power a vehicle such as an over-the-road vehicle (not shown). The engine 10 can be a compression ignition engine, such as a diesel engine, for example. Generally speaking, the SCR system 12 includes a catalyst 20, a reductant supply 22, a reductant injector 24, an electronic control unit 26, and one or more parameters sensors.

The ECU 26 controls delivery of a reductant, such as ammonia, from the reductant supply 22 and into the exhaust system 28 through the reductant injector 24. The reductant supply 22 can include canisters (not shown) for storing ammonia in solid form. In most systems, a plurality of canisters will be used to provide greater travel distance between recharging. A heating jacket (not shown) is typically used around the canister to bring the solid ammonia to a sublimation temperature. Once converted to a gas, the ammonia is directed to the reduction injector 24. The reductant injector 24 is positioned in the exhaust system 28 upstream from the catalyst 20. As the ammonia is injected into the exhaust system 28, it mixes with the exhaust gas and this mixture flows through the catalyst 20. The catalyst 20 causes a reaction between NOₓ present in the exhaust gas and a NOₓ reducing agent (e.g., ammonia) to reduce/convert the NOₓ into nitrogen and water which then passes out of the tailpipe 30 and into the environment. While the SCR system 12 has been described in the context of solid ammonia, it will be appreciated that the SCR system could alternatively use a reductant such as pure anhydrous ammonia, aqueous ammonia or urea, for example.

The ECU 26 controls operation of the SCR system 12, including operation of the reductant injector 24, based on a plurality of operating parameters. In the exemplary embodiment, the operating parameters include intake manifold pressure (IMP), engine speed (N) (i.e., rotational speed), engine load or torque (TQ) and the level of NOₓ in engine's exhaust (Engine Out NOₓ). The intake manifold pressure (IMP) can be determined via a pressure sensor 52 positioned to sense the pressure in the engine's intake manifold and produce a responsive output signal. The engine speed (N) can be determined using a sensor 54 to detect the rotation speed of the engine, e.g., crankshaft rpm. Engine load (TQ) can be based on accelerator pedal position as measured by a sensor 58 or fuel setting, for example. As explained in greater detail, the level of NOₓ in engine's exhaust (Engine Out NOₓ) is estimated by the ECU based on the engine speed (N), load (TQ) and intake manifold pressure (IMP).

In addition to controlling the dosing or metering of ammonia, the ECU 26 can also store information such as the amount of ammonia being delivered, the canister providing the ammonia, the starting volume of deliverable ammonia in the canister, and other such data which may be relevant to determining the amount of deliverable ammonia in each canister. The information may be monitored on a periodic or continuous basis. When the ECU 26 determines that the amount of deliverable ammonia is below a predetermined level, a status indicator (not shown) electronically connected to the controller 26 can be activated.

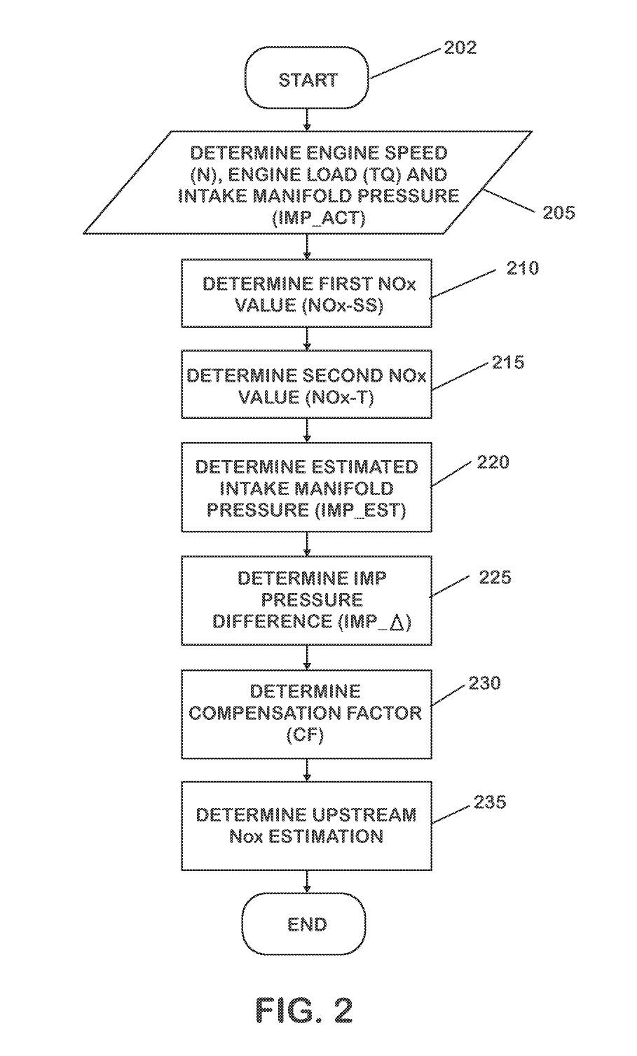

FIG. 2 is a flow chart of an exemplary method 200 for determining the NOₓ level in an engine's exhaust in accordance with certain aspects of the present technology. The method 200 begins in step 202. Control is then passed to the step 205, where the exemplary method determines the engine speed (N), the engine load (TQ) and the actual intake manifold pressure (IMP_ACT), e.g., by reading the output from the sensors 52, 54, 58.

Control is then passed to step 210, where the method 200 determines a first NOₓ value or estimate (NOₓ_{_}SS) as a function of engine speed (N) and engine load (TQ). The first NOₓ estimate (NOₓ_{_}SS) corresponds to the NOₓ output by engine under a first engine operating condition (and at a given speed (N) and load (TQ) combination). In some embodiments, the first operating condition corresponds to substantially “steady state” operation of the engine, i.e., at constant or slowly changing engine speed. In some embodiments, the method 200 determines the first NOₓ estimate (NOₓ_{_}SS) by accessing a look-up table or map that provides an estimate of the NOₓ level produced by the engine at the given engine speed (N) and load (TQ) during the first operating condition (e.g., steady state operation). The look-up table can, for example, be empirically constructed by operating the engine in the first operating condition and measuring actual NOₓ level, i.e., with a NOₓ sensor, at different engine speed and load combinations.

Control is then passed to step 215 where the method determines a second NOₓ value or estimate (NOₓ_{_}T) as a function of engine speed (N) and engine load (TQ). The second NOₓ estimate (NOₓ_{_}T) corresponds to the NOₓ output by the engine during a second operating condition (and at a given engine speed (N) and load (TQ) combination). In some embodiments, the second operating condition corresponds to “transient” operation where engine power is increasing, e.g., during acceleration of a vehicle. In some embodiments, the method 200 determines the second NOₓ value (NOₓ_{_}T) by accessing a look-up table or map that provides an estimate of the NOₓ level produced by the engine at the given engine speed (N) and load (TQ) under the second operating condition (e.g., transient operation).

Next, in step 220 the method 200 determines an estimated intake manifold pressure (IMP_EST) as a function of at least engine speed (N) and torque (TQ). In the exemplary embodiment, the estimated intake manifold pressure (IMP_EST) corresponds to the engine's intake manifold pressure when the engine is under the first operating condition (and at a given engine speed (N) and load (TQ) combination). In some embodiments, the method determines the estimated intake manifold pressure (IMP_EST) by accessing a look-up table or map that provides an estimate of the intake manifold pressure (IMP) at the given engine speed (N) and load (TQ) during the first operating condition (e.g., steady state operation). The look-up table can, for example, be empirically constructed by operating the engine in the first mode and measuring actual intake manifold pressure, i.e., with a sensor, at different engine speed and load combinations.

Control is then passed to step 225 where the method 200 determines a pressure difference (IMP_Δ) between the estimated intake manifold pressure (IMP_EST) and the actual intake manifold pressure (IMP_ACT). Control is then passed to step 230 where the method determines a compensation factor (CF) based on the pressure difference (IMP_Δ) between the estimated and actual intake manifold pressures. According to some embodiments, the compensation factor ranges from 0 when the pressure difference is at first threshold and 1 when the pressure difference is at a second threshold.

Control is then passed to step 235 where the method 200 determines the estimated NOx level being output from the engine (NOₓ_{_}OUT_EST). In some embodiments, the NOₓ output by the engine is determined as a function of the compensation factor and the first and second NOₓ estimates. According to at least some as embodiments of the present technology, the estimated engine out NOₓ (NOₓ_{_}OUT_EST) can be determined in accordance with the following equation.

NOₓ_{_}OUT_EST=(CF•NOₓ_{_}T)+((1−CF)•NOₓ_{_}SS)

The estimated engine at NOₓ (NOₓ_{_}OUT_EST) can be used by the ECU in controlling the SCR system, including controlling the reductant value in order to control dosing of reductant into the exhaust system 28.

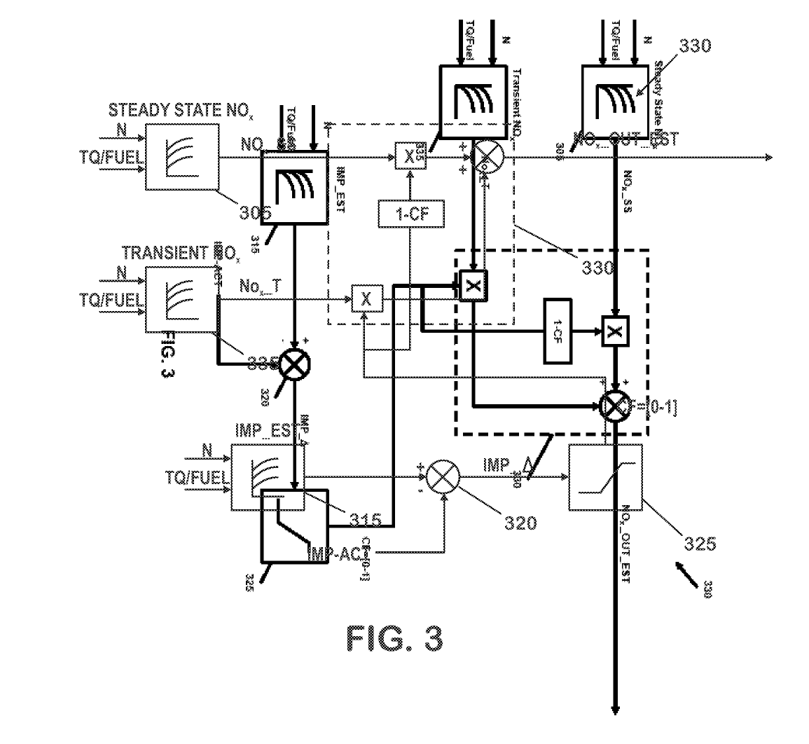

FIG. 3 is a schematic of exemplary control logic 300 for determining the NOₓ level in an engine's exhaust in accordance with certain aspects of the present technology. The control logic includes a first block 305 that determines a first NO value (or estimate) (NOₓ_{_}SS) as a function of at least engine speed (N) and engine load (TQ). The first NOₓ estimate (NOₓ_{_}SS) output by the first logic block 305 corresponds to the NOₓ output by engine under a first engine operating condition (and at a given speed (N) and load (TQ) combination). In some embodiments, the first operating condition corresponds to substantially “steady state” operation of the engine, i.e., at constant or slowly changing engine speed. In at least some embodiments, the control logic 300 determines the first NOₓ value (NOₓ_{_}SS) by accessing a look-up table or map that provides an estimate of the NOₓ level produced by the engine at the given engine speed (N) and load (TQ) during the first operating condition (e.g., steady state operation). The look-up table can, for example, be empirically constructed by operating the engine in the first operating condition and measuring actual NOₓ level, i.e., with a NOₓ sensor, at different engine speed and load combinations.

The control logic 300 also includes a second logic block 310 that determines a second NOₓ value (or estimate) (NOₓ_{_}T) as a function of at least engine speed (N) and engine load (TQ). The second NOₓ estimate (NOₓ_{_}T) output by the second logic block 310 corresponds to the NOₓ output by the engine during a second operating condition (and at a given engine speed (N) and load (TQ) combination). In at least some embodiments, the second operating condition corresponds to “transient” operation where engine power is increasing, e.g., during acceleration of a vehicle. In some embodiments, the control logic 300 determines the second NOₓ value (NOₓ_{_}T) by accessing a look-up table or map that provides an estimate of the NOₓ level produced by the engine at the given engine speed (N) and load (TQ) under the second operating condition (e.g., transient operation). The look-up table can be empirically constructed by operating the engine under the second condition and measuring the actual NOₓ level, i.e., with a sensor, output from the engine at different speed and load combinations.

Control logic 300 also includes a third logic block 315 that determines an estimated intake manifold pressure (IMP_EST) as a function of at least engine speed (N) and torque (TQ). In at least one embodiment, the estimated intake manifold pressure (IMP_EST) corresponds to the engine's intake manifold pressure when the engine under the first operating condition (and at a given engine speed (N) and load (TQ) combination). According to some embodiments, the estimated intake manifold pressure (IMP_EST) corresponds to the engine's intake manifold pressure when the engine is operating at steady state (and at a given engine speed (N) and load (TQ) combination). In some embodiments, the control logic determines the estimated intake manifold pressure (IMP_EST) by accessing a look-up table or map that provides an estimate of the intake manifold pressure (IMP) at the given engine speed (N) and load (TQ) during the first operating condition (e.g., steady state operation). The look-up table can, for example, be empirically constructed by operating the engine in the first operating condition (e.g., steady state operation) and measuring actual intake manifold pressure, i.e., with a sensor, at different engine speed and load combinations.

Control logic includes logic 320 for calculating a pressure difference (IMP_Δ) between the estimated intake manifold pressure (IMP_EST) and the actual intake manifold pressure (IMP_ACT). A fourth logic block 325 determines a compensation factor (CF) as a function of the pressure difference (IMP_Δ) between the estimated and actual intake manifold pressures. According to some embodiments, the compensation factor (CF) ranges from 0 when the pressure difference is at first threshold and 1 when the pressure difference is at a second threshold.

The control logic also includes logic 330 for estimating NOₓ level being output from the engine (NOₓ_{_}OUT_EST) as a function of the compensation factor (CF), the first NOₓ estimate (NOₓ_{_}SS) and the second NOₓ estimate (NOₓ_{_}T). According to at least some embodiments of the present technology, the estimated engine output NOₓ (NOₓ_{_}OUT_EST) can be determined in accordance with the following equation.

NOₓ_{_}OUT_EST=(CF•NOₓ_{_}T)+((1−CF)•NOₓ_{_}SS)

While this disclosure has been described as having exemplary embodiments, this application is intended to cover any variations, uses, or adaptations using the general principles set forth herein. It is envisioned that those skilled in the art may devise various modifications and equivalents without departing from the spirit and scope of the disclosure as recited in the following claims. Further, this application is intended to cover such departures from the present disclosure as come within the known or customary practice within the art to which it pertains. While this disclosure has been described as having exemplary embodiments, this application is intended to cover any variations, uses, or adaptations using the general principles set forth herein. It is envisioned that those skilled in the art may devise various modifications and equivalents without departing from the spirit and scope of the disclosure as recited in the following claims. Further, this application is intended to cover such departures from the present disclosure as come within the known or customary practice within the art to which it pertains.

1. A method for estimating the NOₓ content of exhaust gas produced by an internal combustion engine, the method comprising:
determining a first NOₓ estimate corresponding to the NOₓ level output by the engine during a first engine operating condition;
determining a second NOₓ estimate corresponding to the NOₓ level output by the engine during a second engine operating condition;
determining a compensation factor based on intake manifold pressure;
applying the compensation factor to the first and second NOₓ estimates to arrive at a final NOₓ estimate.

2. A method as set forth in claim 1, wherein the first and second NOₓ estimates are each determined as a function of at least engine speed and torque.

3. The method of claim 2, wherein the first engine operating condition corresponds to substantially steady state engine operation where the engine is operating at a substantially constant speed.

4. The method of claim 2, wherein the second engine operating condition corresponds to a transitory engine operation where engine power is increasing.

5. A method as set forth in claim 1, wherein the step of determining a compensation factor further comprises:
determining an estimated intake manifold pressure as a function of engine speed and torque;
sensing the actual intake manifold pressure; and
determining the compensation factor as a function of the actual and estimated intake manifold pressures.

6. A method as set forth in claim 5, wherein the compensation factor is determined as a function of a difference between the actual and estimated intake manifold pressures.

7. A method as set forth in claim 6, wherein the compensation factor is also a function one or more of exhaust manifold pressure, mass air flow, turbocharger boost, exhaust flow, and combinations thereof.

8. A method as set forth in claim 1, wherein the first and second NOₓ estimates are determined by accessing look up tables.

9. A method as set forth in claim 1, wherein the compensation factor has a value ranging from 0 to 1 and wherein the final NOₓ estimate factor is determined in accordance with the following formula:
NOₓ_{_}OUT_EST=(CF•NOₓ_{_}T)+((1−CF)•NOₓ_{_}SS)
where CF is the compensation factor, NOₓ_{_}SS is the first NOₓ estimate and NOₓ_{_}T is the second NOₓ estimate.

10. A method for estimating the NOₓ content of exhaust gas produced by an internal combustion engine, the method comprising:
determining a steady state NOₓ estimate as a function of at least engine speed and torque, the steady state NOₓ corresponding to the NOₓ level output by the engine during a substantially steady state operation where engine speed and power are substantially constant;
determining a transitory NOₓ estimate as a function of at least engine speed and torque, the transitory NOₓ estimate corresponding to the NOₓ level output by the engine during a transitory operation where engine power is increasing;
determining a compensation factor based on intake manifold pressure;
applying the compensation factor to the steady state and transitory NO estimates to arrive at a final NOₓ estimate, wherein the compensation factor weights the final NOₓ estimate towards the first NOₓ estimate with decreasing intake manifold pressure.

11. A method as set forth in claim 10, wherein the step of determining a compensation factor further comprises:
determining an estimated intake manifold pressure as a function of at least engine speed and torque;
sensing the actual intake manifold pressure; and
determining the compensation factor as a function of a difference between the actual and estimated intake manifold pressures.

12. A method as set forth in claim 11, wherein the compensation factor is also a function one or more of exhaust manifold pressure, mass air flow, turbocharger boost, exhaust flow, and combinations thereof.

13. A method as set forth in claim 11, wherein the compensation factor has a value ranging from 0 to 1 and wherein the final NOₓ estimate is determined in accordance with the following formula:
NOₓ_{_}OUT_EST=(CF•NOₓ_{_}T)+((1−CF)•NOₓ_{_}SS)
where CF is the compensation factor, NOₓ_{_}T is the transient NOₓ estimate and NOₓ_{_}SS is the steady state NOₓ estimate.

14. A method for estimating the NOₓ content of exhaust gas produced by an internal combustion engine, the method comprising:
determining a steady state NOₓ estimate (NOₓ_{_}SS) as a function of at least engine speed and torque, the steady state NOₓ corresponding to the NOₓ level output by the engine during a substantially steady state operation where engine speed and power are substantially constant;
determining a transitory NOₓ estimate (NOₓ_{_}T) as a function of at least engine speed and torque, the transitory NOₓ estimate corresponding to the NOₓ level output by the engine during a transitory operation where engine power is increasing;
determining an estimated intake manifold pressure as a function of at least engine speed and torque;
sensing the actual intake manifold pressure;
determining the compensation factor (CF) as a function of a difference between the actual and estimated intake manifold pressures, wherein the compensation factor has a value ranging from 0 to 1 and increases as the difference between the actual and estimated intake manifold pressures increases;
determining final NOₓ estimate NOₓ_{_}OUT_EST in accordance with the following formula:
NOₓ_{_}OUT_EST=(CF•NOₓ_{_}T)+((1−CF)•NOₓ_{_}SS).
